(19) United States
(12) Patent Application Publication
(10) Pub. No.: US 2011/0008396 A1
(43) Pub. Date: Jan. 13, 2011

(54) COMPOSITIONS AND METHODS FOR TREATING CARDIOVASCULAR CONDITIONS

(75) Inventors: Prabhas Moghe, New Brunswick, NJ (US); Kathryn Uhrich, New Brunswick, NJ (US); Jinzhong Wang, New Brunswick, NJ (US); Nicole Iverson, New Brunswick, NJ (US); Nicole Plourde, New Brunswick, NJ (US)

Correspondence Address: VIKSNINS HARRIS & PADYS PLLP, P.O. BOX 111098, ST. PAUL, MN, 55111-1098, US

(73) Assignee: Rutgers, the Sate University of New Jersey, New Brunswick, NJ (US)

(21) Appl. No.: 12/679,245

(22) PCT Filed: Sep. 22, 2008
(86) PCT No.: PCT/US08/77247

§ 371 Date: Sep. 1, 2010

Related U.S. Application Data
(60) Provisional application No. 60/994,636, filed on Sep. 20, 2007.

(51) Int. Cl. A61K 31/195; A61K 9/00; A61P 9/00; A61P 9/10

(52) U.S. Cl. 424/400; 514/567

(57) ABSTRACT

The invention provides amphiphilic macromolecule encapsulates that are useful for treating cardiovascular diseases including conditions related to or emanating from atherosclerosis.

### PRIORITY OF INVENTION

This application claims the benefit under 35 U.S.C. §119(e) of the filing date of U.S. provisional application Ser. No. 60/994,636, filed 20 Sep. 2007, the disclosure of which is incorporated by reference herein in its entirety.

### BACKGROUND OF THE INVENTION

Atherosclerosis is triggered by interactions between macrophages, smooth muscle cells and their extracellular matrix molecules, subsequent to the pathologic build-up of low density lipoproteins (LDL) within the vascular wall. This condition leads to coronary heart disease, which is the single leading cause of death in America. Elevated plasma levels of LDL lead to the chronic presence of LDL in the arterial wall. There, LDL is modified (oxidized), and capable of activating endothelial cells, which in turn recruit circulating monocytes which infiltrate the vessel wall, differentiate into macrophages, and endocytose oxidized LDL (oxLDL) through scavenger receptor pathways. The LDL interacts with macrophages through various receptors subject to the degree of oxidation (Zhang, H., Y. Yang, et al. (1993), J. Biol. Chem. 268: 5535-5542; and Lougheed, M., E. D. Moore, et al. (1999), Arterioscler Thromb Vasc Biol 19: 1881-1890.). Unoxidized, or native, LDL is internalized primarily by means of the LDL receptor and is controlled by feedback inhibition. OxLDL uptake is mediated by scavenger receptors, which are typically not down-regulated (Goldstein, J. L., Y. K. Ho, et al. (1979), Proceedings of the National Academy of Sciences of the United States of America 76(1): 333-337.). This is a key step in the progression of atherosclerosis and leads to unregulated cholesterol accumulation, and results in the formation of foam cells and the formation of fatty streaks which are the earliest visible atherosclerotic lesions (Brown, M. S. and J. L. Goldstein (1983), Ann. Rev. Biochem. 52: 223-261.). Reverse cholesterol transport (RCT), the transfer of cholesterol from extra-hepatic tissues, like arterial macrophages, to the liver for processing is a pathway for reducing some of the excessive cholesterol accumulation. The shuttle for reverse cholesterol transport, high density lipoprotein (HDL), plays a chief role in the evolution of atherosclerosis (Cuchel, M. and D. J. Rader (2006), Circulation 113(21): 2548-55.).

A number of Liver X receptor (LXR) target genes in macrophages have been linked to the regulation of reverse cholesterol transport, where excess cholesterol is transported to the liver via HDL particles (Geyeregger, R., M. Zeyda, et al. (2006), Cell Mol Life Sci 63(5): 524-39.). LXRs belong to a family of nuclear membrane proteins that become transcriptionally activated through ligand binding (Geyeregger, R., M. Zeyda, et al. (2006), Cell Mol Life Sci 63(5): 524-39.). In addition, LXRs block NF-kB signaling, where NF-kB is required for the induction of TNF-a and IL-6, which are inflammatory cytokines (Joseph, S. B., A. Castrillo, et al. (2003), Nat Med 9(2): 213-9; and Li, Y., R. F. Schwabe, et al. (2005), J Biol Chem 280(23): 21763-72.). Treatment with an LXR agonist has been shown to reduce the formation of foam cells in macrophages by increasing cellular cholesterol efflux and has been shown to reduce lesion formation in apoE^{-/-} and LDLR^{-/-} mice by 50% (Joseph, S. B., E. McKilligin, et al. (2002), Proc Natl Acad Sci USA 99(11): 7604-9.). Additionally, the synthetic agonists T0901317 and GW3965 have been shown to induce expression of LXR related genes in an in vivo mouse model (Repa, J. J., G. Liang, et al. (2000), Genes Dev 14(22): 2819-30; Repa, J. J., S. D. Turley, et al. (2000), Science 289(5484): 1524-9; Schultz, J. R., H. Tu, et al. (2000), Genes Dev 14(22): 2831-8; and Joseph, S. B., E. McKilligin, et al. (2002), Proc Natl Acad Sci USA 99(11): 7604-9.).

Amphiphilic scorpion-like macromolecules (AScMs) have previously been shown to inhibit LDL's uptake by macrophage cells (Chnari, E., L. Tian, et al. (2005), Biomaterials 26: 3749-3758; Chnari, E., J. S. Nikitczuk, et al. (2006), Biomacromolecules 7(2): 597-603; Chnari, E., J. S. Nikitczuk, et al. (2006), Biomacromolecules 7(6): 1796-1805; Wang, J., N. M. Plourde, et al. (2007), Int J Nanomedicine 2(4): 697-705; and International Patent Application Number PCT/US2005/002900). These nanoparticles are ideal for treatment due to their composition of biocompatible components—poly(ethylene glycol) (PEG), music acid and aliphatic acid (Zalipsky, S., N. Mullah, et al. (1997), Bioconjugate Chemistry 8(2): 111-8; and Tian, L., L. Yam, et al. (2004), Macromolecules 37(2): 538-543.). The AScMs form micelles when concentrations are above the critical micelle concentrations (CMCs) (10⁻⁷M) (Tian, L., L. Yam, et al. (2004), Macromolecules 37(2): 538-543.). It has been shown that when the nanoparticles are delivered above the CMC they will bind to macrophage scavenger receptors, thereby decreasing the internalization of hoxLDL by the cells to an appreciable degree (Chnari, E., J. S. Nikitczuk, et al. (2006), Biomacromolecules 7(2): 597-603; and Chnari, E., J. S. Nikitczuk, et al. (2006), Biomacromolecules 7(6): 1796-805.). The hydrophobic core of the nanoparticle has been shown to be useful for drug delivery, showing a high loading efficiency and providing sustained release (Tao, L. and K. E. Uhrich (2006), J Colloid Interface Sci 298(1): 102-10; and International Patent Application Number PCT/US03/17902).

In spite of the above reports, there remains a need for additional compositions and methods that are useful for treating cardiovascular diseases. For example, current cholesterol treatments aim to secondarily manage the localized accumulation of cholesterol (atherogenesis) by reducing systemic levels of cholesterol, which has many side effects (from gastrointestinal complaints to liver enzyme elevation and myopathy) (McKenney, J. M. (2001), Am J Manag Care 7(9 Suppl): S299-306; and DeNoon, D. (2002), WebMD Medical News.). Accordingly, there is a need for new compositions and methods whose mechanisms of action are designed for primarily reducing localized accumulation of cholesterol, for compositions and methods that reduce localized accumulation of cholesterol with fewer side effects, and for compositions and methods that reduce the dose of agent required to reduce localized accumulation of cholesterol.

### SUMMARY OF THE INVENTION

In one embodiment the invention provides a composition of the invention that is a composition comprising a cardiovascular agent and one or more compounds of formula (I):

A-X—Y—Z—R₁ (I)

wherein:

A is H, sulfo-oxy (HOSO₂—O—), —C(═O)N(H)—Rₐ, or —C(═O)O—Rₐ

Rₐ is H, R_{b}, or a (C₁-C₆)alkyl chain, wherein one or more carbon atoms in the alkyl chain is optionally replaced with NH, which chain is optionally substituted with one or more carboxy, sulfo-oxy, amino, or R_{b;}

R_{b} is

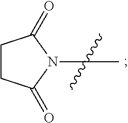

X is a polyol, wherein one or more polyol hydroxyls are substituted by acyl;

Y is —C(═O)—, —C(═S)—, or is absent;

Z is O, S or NH; and

R₁ is a polyether.

The invention also provides a method for inhibiting atherosclerosis or atherosclerotic development in an animal, comprising administering a composition of the invention to the animal (e.g. a mammal such as a human).

The invention further provides the use of a composition of the invention to prepare a medicament useful for treating cardiovascular diseases by inhibiting atherosclerosis or atherosclerotic development in an animal.

In one embodiment the invention provides a therapeutic composition comprising a cardiovascular agent encapsulated within nanoscale assembled polymers (NAPs) comprising a plurality of compounds of formula (I). The present invention also provides methods for the preparation and use of such compositions, as well as compounds of formula (I) for incorporation in such NAPs. The combination of certain amphiphilic nanoscale assembly or particles and GW3965, a liver X receptor (LXR) agonist, has been found to exhibit superior inhibition of the intracellular accumulation of the most atherogenic forms of low density lipoprotein (oxidized LDL, abbreviated as oxLDL, not to be confused with native LDL, denoted as LDL). The combination advantageously provides for the synergistic reduction of oxLDL accumulation and the resultant pro-atherogenic outcomes by counteracting oxLDL uptake via competitive binding of the NAPs to macrophage cell scavenger receptors and simultaneously delivering the LXR agonist intracellularly to promote efflux of internalized oxLDL from the cells. Different compositions of the drug-NAP combination can be realized by altering the ratios of the drug and NAP monomers, as well as altering the chemistry (size, degree of PEGylation, charge presentation, and amphiphilicity) of the nanoscale assemblies of polymers. In one embodiment, the invention has application for the acute treatment of unstable atherosclerotic plaques within the circulation, as well as for the detection and management of plaques to preempt the escalation of myocardial infarction and stroke.

### BRIEF DESCRIPTION OF THE DRAWINGS

FIG. 1 illustrates the ability of 1 cM nanoparticle to inhibit uptake significantly more than other NAPs. THP-1 macrophages were incubated with fluorescently labeled highly oxidized LDL (10 μg/ml) for 24 hours at 37 C and 5% CO₂. All three of the nanoparticles tested were able to significantly inhibit hoxLDL uptake by the cells, but the 1 cM nanoparticle lead to only 27% hoxLDL uptake in comparison to the condition with no nanoparticles present, while the mM and 1 cP reduced uptake to 64% and 45% respectively.

FIG. 2 demonstrates the efflux of hoxLDL from macrophage cells when the model drug, GW3965 is present. The efflux of hoxLDL by THP-1 cells was assayed after incubation with fluorescently labeled hoxLDL (10 ug/mL) for 2 hr at 37 C and 5% CO₂. Excess hoxLDL was removed and NAPs (10⁻⁶M) were added for 5 hours. It is evident that GW3965, when encapsulated in any of the NAPs, lead to a greater efflux of hoxLDL, with the most dramatic efflux seen with the 1 cM NAP.

FIG. 3 illustrates the overall influx/efflux of hoxLDL from macrophage cells when the model drug, GW3965, is present. The internalization of hoxLDL by THP-1 cells was assayed after incubation with fluorescently labeled hoxLDL (10 ug/mL) for 24 hours at 37° C. and 5% CO₂. Conditions include NAPs only (10⁻⁶M), GW3965 (10⁻⁸M) non-encapsulated with NAPs (10⁻⁶M), and GW3965 encapsulated within NAPs (10⁻⁶M NAPs and 10⁻⁸M GW3965). It is evident that GW3965 when encapsulated in any of the NAPs lead to a greater reduction in total hoxLDL accumulation, with the most dramatic reduction seen with the 1 cM NAP.

FIG. 4A, FIG. 4B, and FIG. 4C illustrate the upregulation of two atherogenesis related genes, ABCA-1 and NH1R3, in macrophage cells, assayed by incubating hoxLDL (10 ug/mL) with THP-1 macrophages for 24 hours at 37 C and 5% CO₂. Conditions include NAPs only (10⁻⁶M), GW3965 (10⁻⁸M) non-encapsulated with NAPs (10⁻⁶M), and GW3965 encapsulated within NAPs (10⁻⁶M NAPs and 10⁻⁸M GW3965). It was shown that ABCA1 was upregulated to similarly independent of the NAP that was utilized to deliver the ligand, while NH1R3 was upregulated more by 1 cM then by the other two nanoparticles.

FIG. 5 illustrates the ability of GW3965 encapsulated by NAPs to increase high density lipoprotein (HDL) secretion from macrophages. HDL secretion was measured in THP-1 macrophages pre-loaded with 30 ug/ml highly oxidized LDL. After incubation with NAPs with or without GW3965 for 5 hr at 37° C. in the presence of apoA1 the ability of GW3965 encapsulated to increase HDL secretion in THP-1 macrophages was evident and significant.

FIG. 6 illustrates the visual confirmation of the ability of a key NAP configuration to efficiently deliver GW3965 intracellularly to human macrophage cells. Multiphoton images were taken using a Leica TCS SP2 system (Leica Microsystems, Inc., Exton, Pa.) in order to visually confirm the delivery and internalization of GW3965 to THP-1 macrophage cells incubated for 5 hr with 10⁻⁶ M of each polymer. The images show the presence of GW3965 most strongly in cell exposed to GW3965 encapsulated in 1 cM and illustrate the ability of the 1 cM micelle to efficiently deliver GW3965 to THP-1 macrophages. Two representative images of this condition are shown in the top row above. In contrast, the control images of cells alone, cells with NAP alone, and cells incubated with the drug in the absence of NAP formulation, show no drug uptake (see bottom row).

### DETAILED DESCRIPTION OF THE INVENTION

### Definitions

The generic phrase “low-density lipoprotein (LDL)” usually encompasses “unoxidized or native LDL,” “weakly oxidized LDL” and “oxidized LDL” but functionally, these terminologies have distinct connotations within the invention. Thus, “unoxidized low-density lipoprotein” refers to a native LDL, e.g., an LDL that has the characteristics of an LDL that is recognized by a native LDL receptor. In contrast, an “oxidized LDL (ox-LDL)” is a modified LDL recognized by scavenger receptors. By the phrase “weakly oxidized low-density lipoprotein (LDL)” is meant a mildly or partially oxidized LDL. Both unoxidized and weakly oxidized LDL have relatively high localized positive charges, e.g., due to unmodified Lys and Arg residues on apolipoprotein B-100 (ApoB-100) (LDL have a single Apo B-100 molecule on their surface) as compared to oxidized LDL. See, for example, Chnari et al., Biomaterials, 26: 3749-3758 (2005). LDLs bind to proteoglycans (PGs), the major low density lipoprotein (LDL)-retentive matrix molecules within the vascular intima are proteoglycans. LDL binding to PGs modifies the LDL surface, rendering the LDL susceptible to oxidation induced by Cu²⁺ and macrophages. The oxidative modification of LDL lowers its localized positive charge relative to native LDL, thus reducing the affinity of LDL for anionically charged PGs. The increase in the net negative charge on oxidized LDL also leads to the reduced recognition of oxidized LDL by the classical LDL receptor, and increased recognition by the scavenger receptors on macrophages in the intima. The abbreviation of “HDL” indicates high density lipoprotein (not to be confused with “Hox-LDL” or “ox-LDL”, which stands for highly oxidized low density lipoproteins).

By “inhibition of atherosclerotic development” is meant the suppression of the development, progression and/or severity of atherosclerosis, a slowly progressive disease characterized by the accumulation of cholesterol within the arterial wall, e.g. by inhibiting, preventing or causing the regression of an atherosclerotic plaque.

As used herein the term “polyol” includes straight chain and branched chain aliphatic groups, as well as mono-cyclic and poly-cyclic aliphatics, which may be substituted with two or more hydroxy groups. A polyol typically about 2 carbons to about 20 carbons (C₂-C₂₀); preferably about 3 carbons to about 12 carbons (C₃-C₁₂); and more preferably about 4 carbons to about 10 carbons (C₄-C₁₀). A polyol also typically comprises from about 2 to about 20 hydroxyl; preferably about 2 to about 12 hydroxyl; and more preferably about 2 to about 10 hydroxyl. A polyol also optionally may be substituted on a carbon atom with one or more, e.g. 1, 2, or 3, carboxyl (COOH), which may be used to link the polyol to a polyether or to A (e.g. through an ester or amide linkage) in one embodiment of the compound of formula (I).

One specific polyol comprises a mono- or di-carboxyilic acid comprising about 1 to about 10 carbon atoms (C₁-C₁₀) and may be substituted with 1 to about 10 hydroxyl. The mono- or di-carboxylic acid may be a straight chained or branched chained aliphatic, or a mono-cyclic or poly-cyclic aliphatic compound. Suitable dicarboxylic acids include mucic acid, malic acid, citromalic acid, alkylmalic acid, hydroxy derivatives of glutaric acid, and alkyl glutaric acids, tartaric acid, citric acid, hydroxy derivatives of rumadic acid, and the like. Suitable monocarboxylic acids include 2,2-(bis(hydroxymethyl)propionic acid, and N-[tris(hydroxymethyl)methyl]glycine (tricine). Other mono and di-carboxylic acids, however, are also suitable for use with this invention.

Another specific polyol comprises a “saccharide,” e.g. monosaccharides, disaccharides, trisaccharides, polysaccharides and sugar alcohols, among others. The term saccharide includes glucose, sucrose, fructose, ribose, and deoxy sugars such as deoxyribose, and the like. Saccharide derivatives may be prepared by methods known to the art. Examples of suitable mono-saccharides are xylose, arabinose, and ribose. Examples of di-saccharides are maltose, lactose, and sucrose. Examples of suitable sugar-alcohols are erythritol and sorbitol. Other mono- and di-saccharide, saccharide derivatives, and sugar alcohols, however, are also suitable.

As used herein, the term polyether includes poly(alkylene oxides) of for example, about 2 to about 150 repeating units. Typically, the poly(alkylene oxides) comprises about 50 to about 110 units, which may include the same or different residues, e.g. repeating or non-repeating units. The alkylene oxide units may comprise about 2 to about 20 carbon atoms, i.e. straight or branched (C₂-C₂₀) alkyl, or about 2 to about 10 carbon atoms (C₁-C₁₀). Poly(ethylene glycol) (PEG) is one specific polyether. Alkoxy-, amino-, carboxy-, carboxymethoxy-, sulfo-oxy, and sulfo-terminated poly(alkylene oxides) are all included. In one embodiment the polyether is methoxy-terminated or carboxymethoxy terminated.

One preferred polyether comprises the chemical structure

R₅—(R₆—O—)ₐ—R₆-Q- (II),

wherein

R₅ comprises straight or branched (C₁-C₂₀) alkyl,

—OH, —OR₇, —NH₂, —NHR₇, —NHR₇R₈, —CO₂H, —SO₃H, —O—SO₃H, —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—;

R₆ straight or branched divalent (C₂-C₁₀) alkylene;

each R₇ and R₈ comprise, independently, straight or branched (C₁-C₆)alkylene;

Q comprises —O—, —S—, or —NR₇; and

a is an integer from 2 to 150, inclusive.

Another specific polyether is a methoxy terminated polyethylene glycol or a carboxymethoxy terminated polyethylene glycol.

In a compound of this invention a poly(alkylene oxide) may be linked to a polyol, for example, through an ether, thioether, amine, ester, thioester, thioamide, or amide linkage. In one specific embodiment the poly(alkylene oxide) may be linked to a polyol by an ester or amide linkage.

The term acyl includes fatty acid residues. As used herein, the term “fatty acid” includes fatty acids and fatty oils as conventionally defined, for example, long-chain aliphatic acids that are found in natural fats and oils. Fatty acids typically comprise about 2 to about 24 carbon atoms (C₂-C₂₄ fatty acids), or about 6 to about 18 carbon atoms (C₆-C₁₈ fatty acids). The term “fatty acid” encompasses compounds possessing a straight or branched aliphatic chain and an acid group, such as a carboxylate, sulfonate, phosphate, phosphonate, and the like. The “fatty acid” compounds are capable of “esterifying” or forming a similar chemical linkage with hydroxy groups on the polyol. Examples of suitable fatty acids include caprylic, capric, lauric, myristic, myristoleic, palmitic, palmitoleic, stearic, oleic, linoleic, eleostearic, arachidic, behenic, erucic, and like acids. Fatty acids may be derived from suitable naturally occurring or synthetic fatty acids or oils, may be saturated or unsaturated, and optionally may include positional and/or geometric isomers. Many fatty acids or oils are commercially available or may be readily prepared or isolated using procedures known to those skilled in the art.

In one embodiment, the compound of formula (I) is:

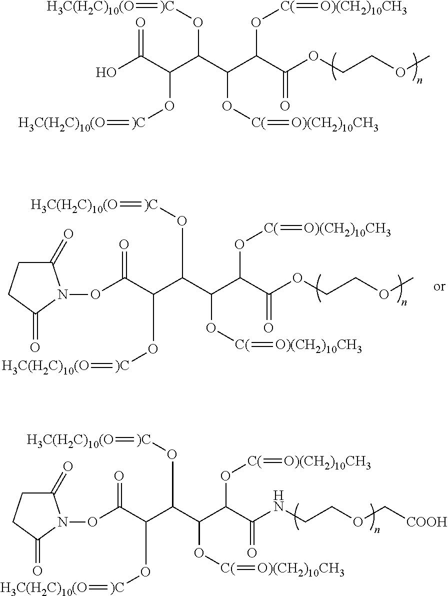

wherein n is 80-200. In another embodiment, n is 100-180. In another embodiment, n is 110-115.

The nature of the “linker” is not critical provided it does not interfere with the desired function of the compound or conjugate. For example, the linker can include a straight or branched carbon chain having from about one to about 20 carbon atoms; the carbon chain can optionally be saturated or unsaturated and can optionally be interrupted with one or more heteroatoms (e.g. oxygen, sulfur, or nitrogen). In one embodiment, the linker has from about 2 to about 10 carbon atoms.

In one embodiment of the invention, the diameter of the NAP comprised of a plurality of compounds of formula (I) is less than 250 nm. In another embodiment of the invention, the diameter of the NAP is less than 150 nm. In another embodiment of the invention, the diameter of the NAP is less than 100 nm. In another embodiment of the invention, the diameter of the NAP is less than 80 nm. In another embodiment of the invention, the diameter of the NAP is less than 70 nm. In another embodiment of the invention, the diameter of the NAP is less than 60 nm. In another embodiment of the invention, the diameter of the NAP is less than 50 nm. In another embodiment of the invention, the diameter of the NAP is less than 40 nm. In another embodiment of the invention, the diameter of the NAP is less than 30 nm. In another embodiment of the invention, the diameter of the NAP is less than 25 nm. In another embodiment of the invention, the diameter of the NAP is less than 20 nm.

In another embodiment of the invention, the diameter of the NAP is greater than 5 nm. In another embodiment of the invention, the diameter of the NAP is greater than 10 nm. In another embodiment of the invention, the diameter of the NAP is greater than 15 nm. In another embodiment of the invention, the diameter of the NAP is greater than 20 nm. In another embodiment of the invention, the diameter of the NAP is greater than 25 nm. In another embodiment of the invention, the diameter of the NAP is greater than 30 nm. In another embodiment of the invention, the diameter of the NAP is greater than 35 nm. In another embodiment of the invention, the diameter of the NAP is greater than 40 nm. In another embodiment of the invention, the diameter of the NAP is greater than 45 nm. In another embodiment of the invention, the diameter of the NAP is greater than 50 nm. In another embodiment of the invention, the diameter of the NAP is greater than 55 nm.

In certain embodiments, four criteria may be employed to aid in the design of compounds of formula (I) for incorporation into compositions of the invention. First, a tunable hydrophilic-lipophilic balance (HLB) is desired to match the compounds with the hydrophobicity of the cardiovascular agent to optimize drug-carrier interactions. Second, polymer systems themselves should not cause any undesirable biological complications, such as toxicity and immunogenicity. See, e.g. Moghimi, S. M., Adv. Drug Delivery Rev. 1995, 17, 1. Third, the polymers should typically be biodegradable and easily excretable by living systems. Fourth, the inclusion of biological functionality significantly aids in the selective biomedical applications.

### Cardiovascular Agents and Compounds

As used herein, the term cardiovascular agent includes Anticoagulants, Antiplatelet Agents, Angiotensin-Converting Enzyme (ACE) Inhibitors, Angiotensin II Receptor Blockers, Beta Blockers, Diuretics, Vasodilators Digitalis Preparations, Statins, and Liver X Receptor Ligands.

In one specific embodiment, the cardiovascular agent is a 20S Proteasome Inhibitor, 3-Hydroxyl-3-Methylglutaryl Coenzyme A (HMG-CoA) Reductase Inhibitor, 3-KetoAcyl-Coa-Thiolase (3-KAT) Inhibitor, 5-Lipoxygenase-Activating Protein (FLAP) Inhibitor, Adenosine A1 Receptor (ADORA) Angonist, Adenosine Deaminase (ADA) Inhibitor, Aldosterone Receptor Agonist, Alpha Adrenergic Receptor Agonist, Alpha Adrenergic Receptor Antagonist, Beta Adrenergic Receptor Agonist, Beta Adrenergic Receptor Antagonist, Dopamine D2 Receptor Agonist, Muscarinic Receptor Antagonist, Calcium Channel Blocker, Potassium Channel Blocker, Cardiac Myosin Activator, Complement C1 Esterase Inhibitor, Corticotropic Releasing Factor Receptor Agonist, Cyclooxygenase-1 (COX-1) Inhibitor, Cyclooxygenase-2 (COX-2) Inhibitor, Dopamine Receptor Agonist, Endothelial Nitric Oxide Synthase (eNOS) Enhancer, Endothelin Receptor Antagonist, Fibroblast Growth Factor Receptor Tyrosine Kinase Activator, Factor IX Inhibitor, Gap Junction Opener, Glycoprotein (GP) IIb-IIIa Receptor Antagonist, Guanylyl Cyclase (GC) Activator, Thromboxane A2 Synthesis Inhibitor, Hepatocyte Growth Factor Receptor Agonist, Human Leukocyte Elastase Inhibitor, Hyperpolarization Activated Cyclic Nucleotide-Gated Potassium Channel Blocker, Inducible Nitric Oxide Synthase Inhibitor, Insulin-Like Growth Factor 1 Receptor Tyrosine Kinase Activator, L-3,4-Dihydroxyphenylalanine Decarboxylase Inhibitor, Mannan-Binding Lectin Serine Peptidase Inhibitor, Mast Cell Chymase Inhibitor, Matrix Metalloproteinase Inhibitor, Monoamine Oxidase Inhibitor, N-Methyl-D-Aspartate (NMDA) Receptor Antagonist, Natriuretic Peptide Receptor Agonist, p38 Mitogen-Activated Protein (MAP) Kinase Inhibitor, Peroxisome Proliferator-Activated Receptor-Alpha (PPAR-Alpha) Agonist, Phosphodiesterase Inhibitor, Platelet-Activating Factor (PAF) Inhibitor, Tissue Plasminogen Activator (tPA) Stimulant, Poly(ADP-ribose) polymerase (PARP) Inhibitor, Sodium Channel Blocker, Potassium Channel Opener, Progesterone Receptor (PR) Agonist, Prostaglandin Receptor Agonist, Prostaglandin Synthesis Stimulator, Protein Kinase C-delta (PKC-delta), Inhibitor, S100B Protein Synthesis Inhibitor, Secretory Phospholipase A2 (sPLA2) Inhibitor, Serine Protease Inhibitor, Sodium Hydrogen Exchange (NHE) Inhibitor, Sodium-Calcium Exchange Inhibitor, Sodium-Potassium ATPase Inhibitor, Thromboxane A2 (TXA2) Synthesis Inhibitor, Vascular Endothelial Growth Factor (VEGF) Inducer, Xanthine Oxidase (XO) Inhibitor, Angiotensin Receptor Blocker, Angiotensin Converting Enzyme Inhibitor, or Renin Inhibitor.

In one specific embodiment, the cardiovascular agent is Dalteparin (Fragmin), Danaparoid (Orgaran), Enoxaparin (Lovenox), Heparin (various), Tinzaparin (Innohep), Warfarin (Coumadin), Aspirin, Ticlopidine, Clopidogrel, Dipyridamole, Benazepril (Lotensin), Captopril (Capoten), Enalapril (Vasotec), Fosinopril (Monopril), Lisinopril (Prinivil, Zestril), Moexipril (Univasc), Perindopril (Aceon), Quinapril (Accupril), Ramipril (Altace), Trandolapril (Mavik), Candesartan (Atacand), Eprosartan (Teveten), Irbesartan (Avapro), Losartan (Cozaar), Telmisartan (Micardis), Valsartan (Diovan), Acebutolol (Sectral), Atenolol (Tenormin), Betaxolol (Kerlone), Bisoprolol/hydrochlorothiazide (Ziac), Bisoprolol (Zebeta), Carteolol (Cartrol), Metoprolol (Lopressor, Toprol XL), Nadolol (Corgard), Propranolol (Inderal), Sotalol (Betapace), Timolol (Blocadren), Amlodipine (Norvasc, Lotrel), Bepridil (Vascor), Diltiazem (Cardizem, Tiazac), Felodipine (Plendil), Nifedipine (Adalat, Procardia), Nimodipine (Nimotop), Nisoldipine (Sular), Verapamil (Calan, Isoptin, Verelan), Amiloride (Midamor), Bumetanide (Bumex), Chlorothiazide (Diuril), Chlorthalidone (Hygroton), Furosemide (Lasix), Hydrochlorothiazide (Esidrix, Hydrodiuril), Indapamide (Lozol), Spironolactone (Aldactone), Isosorbide dinitrate (Isordil) Nesiritide (Natrecor), Hydralazine (Apresoline), a Nitrate, Minoxidil, Lanoxin, Lipitor, Crestor, Nicotinic acid (niacin), Gemfibrozil or Clofibrate.

In one specific embodiment, the cardiovascular agent is an agent that modulates cholesterol and lipid metabolism (e.g. statins, resins, nicotinic acid (niacin), gemfibrozil and clofibrate).

In one specific embodiment, the cardiovascular agent is an agent that modulates atherogenesis, the intracellular accumulation of cholesterol.

In one specific embodiment, the cardiovascular agent is a Liver X receptor ligand.

In one specific embodiment, the cardiovascular agent is Diepoxycholesterol, T0901317, GW3965, or 24(S),25-Epoxycholesterol.

### Dosages and Routes of Administration

The compositions of the invention may be formulated as pharmaceutical compositions, and may be administered to a mammalian host, such as a human patient, in a variety of forms adapted to the chosen route of administration, i.e., orally or parenterally, by intravenous, intramuscular, topical, subcutaneous, or other routes. Thus, the compositions of the invention may be systemically administered, e.g., orally, in combination with a pharmaceutically acceptable vehicle such as an inert diluent. They may be incorporated directly with the food of the patient's diet. For oral therapeutic administration, the compositions of the invention may be used in the form of elixirs, syrups, and the like.

The compositions may also contain a sweetening agent such as sucrose, fructose, lactose or aspartame or a flavoring agent such as peppermint, oil of wintergreen, or cherry flavoring may be added. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the compositions of the invention can be formulated into sustained-release preparations and devices.

The compositions of the present invention can be administered to a patient by any of a number of means known in the art, including but not limited to catheterization-accompanying injections for acute treatment and drug-eluting stents for treatment of sustained or chronic conditions.

The compositions of the invention may also be administered intravenously or intraperitoneally by infusion or injection, among many other routes. Solutions may be prepared, for example, in water. However, other solvents may also be employed. Under ordinary conditions of storage and use, these preparations may contain a preservative to prevent the growth of microorganisms, and other formulation ingredients as is known in the art.

The pharmaceutical dosage forms suitable for injection or infusion should be preferably sterile, fluid and stable under the conditions of manufacture and storage. The prevention of the action of microorganisms may be brought about by various antibacterial and antifungal agents, for example, parabens, chlorobutanol, phenol, sorbic acid, thimerosal, and the like. Others are also suitable. In many cases, it may be preferable to include isotonic agents, for example, sugars, buffers or sodium chloride.

Sterile injectable solutions may be prepared by incorporating the compositions of the invention in the required amount into an appropriate solvent or medium with various other ingredients, e.g., those enumerated above, as needed, which may be followed by sterilization.

The dose and method of administration will vary from animal to animal and be dependent upon such factors as the type of animal being treated, its sex, weight, diet, concurrent medication, overall clinical condition, the particular therapeutic agent employed, the specific use for which the agent is employed, and other factors which those skilled in the relevant field will recognize.

Therapeutically effective dosages may be determined by either in vitro, ex vivo, or in vivo methods, in accordance with the intended application. For each particular dosage form of the present invention, individual determinations may be made by an artisan to determine the optimal dosage required. The range of therapeutically effective dosages will naturally be influenced by the route of administration, the therapeutic or diagnostic objective, and the condition of the patient. The determination of effective dosage levels, that is the dosage level necessary to achieve a desired result, will be within the ambit of one skilled in the art. Typically, applications of an agent such as the one of this invention are commenced at low dosage levels, with dosage levels being increased until the desired effect is achieved.

A typical dosage might range from about 0.001 mg to about 1,000 mg of agent per kg of animal weight (mg/kg). Preferred dosages range from about 0.01 mg/kg to about 100 mg/kg, and more preferably from about 0.10 mg/kg to about 20 mg/kg. Advantageously, the dosage forms of this invention may be administered, for example, as a single dose, or several times per day, and other dosage regimens may also be useful. The period of time during which the present product may be administered may vary with the intended application. For acute instances, the administration or application may be conducted for short periods of time, e.g. a few days to one or more weeks or months. For chronic problems, the administration or application may be conducted for even longer periods of time, up to one or more years, or for life, with appropriate monitoring.

The following non-limiting examples set forth hereinbelow illustrate certain aspects of the invention. All parts and percentages are by weight unless otherwise noted and all temperatures are in degrees Celsius.

PEG's were obtained from Shearwater Polymers (Birmingham, Ala.) and used without further purification. All other chemicals were obtained from Aldrich (Milwaukee, Wis.), and used without further purification. Analytical grade solvents were used for all the reactions. Methylene chloride, tetrahydrofuran (THF), triethylamine (TEA) and dimethylsulfoxide (DMSO) were distilled. 4-(dimethylamino)pyridinium p-toluenesulfonate (DPTS) was prepared as described by J. S. Moore, S. I. Stupp Macromolecules 1990, 23, 65. ¹H-NMR and spectra were recorded on a Varian 200 MHz or 400 MHz spectrometer. Samples (˜5-10 mg/ml) were dissolved in CDCl₃ or THF-d₄, with the solvent used as an internal reference. IR spectra were recorded on a Mattson Series spectrophotometer by solvent casting samples onto a KBr pellet. Thermal analysis data were determined on a Perkin-Elmer Pyris 1 DSC system, samples (˜10 mg) were heated under dry nitrogen gas. Data were collected at heating and cooling rates of 5° C./min. Gel permeation chromatography (GPC) was performed on a Perkin-Elmer Series 200 LC system. Dynamic laser scattering (DSL) measurements were carried on NICOMP particle sizing systems.

The invention will now be illustrated by the following non-limiting Examples.

### Examples

### Example 1

### Cell Culture

THP-1, human monocytes, are grown in suspension, with RPMI medium containing 0.4 mM Ca²⁺ and Mg²⁺, (ATCC) and supplemented with 10% FBS, in an incubator with 5% CO₂ at 37 C and split every four days through centrifugation. The cells are seeded at a concentration of 100,000 cells/cm² to continue monocyte cultures and to differentiate the cells into macrophage cells. Once the monocytes have been differentiated into macrophage cells they will become adherent and can be tested. The macrophage cells can not be propagated and will be used within a week of differentiation.

### LDL Oxidation

Highly oxidized LDL will be oxidized within five days of each experiment. BODIPY-labeled and unlabeled human plasma derived LDL (Molecular Probes, OR) is oxidized by 18 hours of incubation with 10 uM CuSO₄ (Sigma) at 37 C with 5% CO₂. (Oorni 1997; Chang 2001) The oxidation will be stopped with 0.01% w/v EDTA after the 18 hours.

### Nanoscale Assembled Polymer (NAP) Synthesis

Compounds of formula (I) can be prepared as described in International Patent Application Number PCT/US03/17902 and International Patent Application Number PCT/US2005/002900)

Synthesis of 1 cM. Polymer was prepared as previously described by Tian, L., L. Yam, et al. (2004), Macromolecules 37(2): 538-543). Briefly, mucic acid was esterified with lauroyl chloride in the presence of zinc chloride to obtain the mucic acid derivative, which was then esterified with hydroxy-poly (ethylene glycol) (5 kDa) with DCC as the dehydrating reagent and DPTS as the catalyst to obtain the desired product. (Tian 2004)

Synthesis of MM. 1 cM was esterified with N-hydroxysuccinimide (4 eqv.) with DCC (1.5 eqv.) as the dehydrating reagent in anhydrous CH₂Cl₂ and DMF. The reaction was allowed to proceed for 24 hours at room temperature under argon gas before being washed with 0.1 N HCl (1×) and 50:50 brine/H₂O (2×), dried, and concentrated. The desired product was then precipitated from CH₂Cl₂ by addition of 10-fold diethyl ether. Yield: 90%. ¹H-NMR (CDCl₃): 5.77 (m, 2H, CH), 5.05 (m, 2H, CH), 4.21 (m, 2H, CH₂), 3.63 (m, ˜0.45 kH, CH₂), 3.38 (s, 3H, CH₃), 2.82 (s, 4H, CH₂), 2.45 (m, 4H, CH₂), 2.30 (m, 4H, CH₂), 1.60 (m, 8H, CH₂), 1.26 (m, 64H, CH₂), 0.88 (t, 12H, CH₃). GPC: M_{w}=6.0 kDa

Synthesis of 1 cP. This polymer was prepared as previously described (Chnari, E; Nikitczuk, et al., Biomacromolecules, 7 (7) 1796-1805 (2006)). Briefly, the carboxylic acids of the mucic acid derivative described above were esterified with N-hydroxysuccinimide with DCC as the dehydrating reagent in anhydrous CH₂Cl₂ and DMF. This product was then coupled with a heterobifunctional PEG, H₂N-PEG-COOH (Mw=5 kDa), in CH₂Cl₂ and triethylamine to yield the desired product.

### Polymer Characterization

Proton nuclear magnetic resonance (¹H-NMR) spectra of the products were obtained using a Varian 400 MHz or 500 MHz spectrophotometer. Samples were dissolved in chloroform-d, with a few drops of dimethyl sulfoxide-d₆ if necessary, with tetramethylsilane as an internal reference. Molecular weights (Mw) were determined using gel permeation chromatography (GPC) with respect to polyethylene glycol standards (Sigma-Aldrich) on a Waters Stryagel® HR 3 THF column (7.8×300 mm). The Waters LC system (Milford, Mass.) was equipped with a 2414 refractive index detector, a 1515 isocratic HPLC pump, and 717plus autosampler. An IBM ThinkCentre computer with Waters Breeze Version 3.30 software installed was used for collection and processing of data. Samples were prepared at a concentration of 10 mg/mL in tetrahydrofuran, filtered using 0.45 μm pore size nylon or poly(tetrafluoroethylene) (PTFE) syringe filters (Fisher Scientific) and placed in sample vials to be injected into the system.

### Encapsulation of Drug/Agent/Ligand

A model drug, GW3965, which is a ligand for the liver-X-receptors (LXR) was selected for encapsulation within the NAPs.

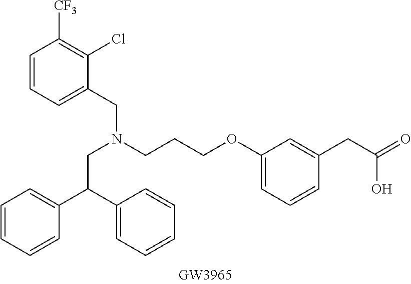

GW3965 solution (1.0 mg/mL) in dichloromethane (CH₂Cl₂) is prepared by combining 5.0 mL CH₂Cl₂ to GW3965 (5.0 mg) (Sigma Aldrich). One equivalent of triethylamine (1.2 μL, 0.88 mg) is added to neutralize the HCl on the GW3965, making it soluble in CH₂Cl₂. 1 cM nanoparticle solution (2.0 mg/mL) is made by dissolving 0.029 g 1 cM in 14.5 mL of ultrapure water and gently stirring for 60 minutes until all solid is dissolved.

To create a 1:100 ratio of drug:polymer (wt/wt), 290 μL GW3965 solution in CH₂Cl₂ is added drop-wise to 14.5 mL of 2.0 mg/mL 1 cM solution. The mixture is then covered and stirred at room temperature for 12-24 hours. The solution should then be stirred uncovered for another 24 hours to allow the CH₂Cl₂ to completely evaporate. The resulting aqueous solution is filtered by vacuum using a cellulose acetate membrane (8 μm pore size) to remove any unbound (or non-encapsulated) drug. The solution is then brought up to a final volume of 50 mL (by adding ultrapure water) and used within one week. Following this method the NAP is 1*10⁻⁴ M and the concentration of the GW3965 is 1*10⁻⁵ M, however this method can also be used for the encapsulation of higher concentrations of GW3965 (e.g. up to at least a 1:10 drug:polymer (wt/wt) ratio).

To ensure that GW3965 is encapsulated by the NAPs, the solution is tested by UV-Vis Spectrophotometry. GW3965 alone absorbs light at 270 nm while a sample of encapsulated GW3965 does not because the GW3965 within the core is shielded by the micelle from UV light. Therefore, a sample of the encapsulated solution is analyzed to ensure that a GW3965 peak does not appear. This sample is then diluted with dimethylacetamide (DMA) in a 1:1 ratio (to disrupt the micelles, releasing the encapsulated GW3965). The absorbance peak at 270 nm is seen in the disrupted sample.

### Sizing

Dynamic light scattering (DLS) analyses were performed using a Malvern Instruments Zetasizer Nano ZS-90 instrument (Southboro, Mass.), with reproducibility being verified by collection and comparison of sequential measurements. NAP solutions at a concentration of 1 mg/mL were prepared using picopure water. Measurements were performed at a 90° scattering angle at 25° C. Z-average sizes of polymers in solution were collected and analyzed.

### UV

UV absorption data were collected on a Beckman DU®520 General Purpose UV/Vis Spectrophotometer. Data was collected of samples in water and samples diluted with 50% DMSO to disrupt the NAPs.

### LDL Influx

The internalization of hoxLDL by macrophage cells was assayed by incubating fluorescently labeled hoxLDL (10 ug/mL) with cells for 24 hours at 37° C. and 5% CO₂. The different conditions that the cells were exposed to include a control condition with only RMPI medium, a NAP alone condition, with the NAPs at the concentration of 1*10⁻⁶M, a GW3965 and NAP condition in which a non-encapsulated ligand at 1*10⁻⁸M is delivered to the cells in conjuction with NAPs at 1*10⁻⁶M, and an encapsulated ligand condition in which the GW3965 is encapsulated by the different NAPs and are at 1*10⁻⁶M for the NAPs and 1*10⁻⁸M for the ligand. The cells were then be washed twice with PBS and imaged. The images were analyzed with Image Pro Plus 5.1 software (Media Cybernetics, San Diego, Calif.) and normalized with cell number before being compared to the control sample where no NAPs or ligand was added.

### LDL Efflux

The efflux of hoxLDL by macrophage cells was assayed by incubating fluorescently labeled hoxLDL (10 ug/mL) with cells for 2 hours at 37 C and 5% CO₂. The excess hoxLDL was then removed from all conditions and the different test conditions were added and incubated for 5 hours at 37° C. and 5% CO₂. The different conditions that the cells were exposed to include a control condition with only RMPI medium, a GW3965 and NAP condition in which a non-encapsulated ligand at 1*10⁻⁸M is delivered to the cells in conjunction with NAPs at 1*10⁻⁶M, and an encapsulated ligand condition in which the GW3965 is encapsulated by the different NAPs and are at 1*10⁻⁶M for the NAPs and 1*10⁻⁸M for the ligand. The cells were then washed twice with phosphate buffered saline (PBS) and imaged. The images were analyzed with Image Pro Plus 5.1 software (Media Cybernetics, San Diego, Calif.) and normalized with cell number and compared to the control sample wherein no NAPs or ligand was administered.

mRNA Expression of Key Genes

The RNA of the samples was extracted with the RNeasy Mini kit from Qiagen. Briefly, cells were lysed with Betamarceptaethenol and QiaShredder before extraction of and purification of the samples. DNA expansion was conducted with Quantitative RT-PCR conducted on a Roche light cycler with B-actin as a housekeeping gene.

### Efflux of High Density Lipoproteins (HDL)

In brief, differentiated THP-1 macrophages were pre-incubated with 30 μg/ml highly oxidized LDL at 37° C. in serum-free RPMI 1640 for 24 hr. Next, NAPs at 10⁻⁶ M and 10 ug/mL ApoA1 with or without 10⁻⁷ M GW3965 were added to each well and incubated for a further 24 hours. The cell medium was then removed for analysis using the Biovision HDL and LDL/VLDL Cholesterol Quantification kit (BioVision, CA). The remaining macrophages were lysed using 0.03 g sodium dodecyl sulfate (SDS) in 30 ml sodium hydroxide (NaOH, 0.1N). The protein content was measured with the Modified Lowry protein assay (Pierce, Ill.) and the HDL secretion results were normalized per mg cell protein.

### GW Internalization Study

In brief, differentiated THP-1 macrophages were incubated with NAPs at 10⁻⁶ M and/or 10⁻⁷M GW3965 for 5 hr in serum-free RPMI at 37° C. Cells were washed and fixed and multiphoton imaging to detect internalized GW3965 was performed on a Leica TCS SP2 system (Leica Microsystems, Inc., Exton, Pa.). The cells were illuminated using a titanium: sapphire femtosecond laser with a tunable wavelength from 780 nm to 920 nm (Mai Tai, repetition rate 80 Mhz, 100 fs pulse duration, 800 mW) and 470-500 nm emission.

### Cell Recovery

The recovery of macrophage cells after the excess internalization of hoxLDL was determined to create an in vitro model that more closely models an in vivo disease condition. Macrophage cells were incubated with fluorescently labeled hoxLDL (10 ug/mL) for 2 hours at 37° C. and 5% CO₂. The excess hoxLDL solution was then removed from all conditions and the different test conditions which now all include 5% FBS serum were added and incubated for 24 hours at 37° C. and 5% CO₂. The different conditions that the cells were exposed to include a control condition with only RMPI medium, a NAP alone condition with the NAPs at the concentration of 1*10⁻⁶M, a GW3965 and nanoparticle condition in which a non-encapsulated ligand at 1*10⁻⁸M is delivered to the cells in conjunction with NAPs at 1*10⁻⁶M, and an encapsulated ligand condition in which the GW3965 is encapsulated by the different nanoparticles and are at 1*10⁻⁶M for the NAPs and 1*10⁻⁸M for the ligand. The cells were then washed twice with PBS and imaged. The images were analyzed with Image Pro Plus 5.1 software (Media Cybernetics, San Diego, Calif.) and normalized with cell number and compared to the control sample where no NAPs or ligand was administered.

### Results

The three amphiphilic macromolecules (AMs) shown in Scheme 1, were synthesized to investigate their ability to deliver a hydrophobic ligand to THP-1 macrophage cells. Initially, the ability of the NAPS to encapsulate the LXR ligand, GW3965, was determined using UV absorption. All NAPS were able to encapsulate GW3965 at high concentrations, as determined by the absence of an absorption peak at 270 nm where GW3965 is known to absorb. The absence of this peak is due to polymer shielding of GW3965 from UV irradiation. To show that the GW3965 was indeed encapsulated and not simply removed upon filtering the solutions, the micelles were disrupted with DMSO allowing them to release the encapsulated GW. Subsequent collection of UV data showed the presence of an absorption peak at 270 nm, the wavelength at which GW3965 absorbs. To ensure no aggregation upon encapsulation, the sizes of the NAP micelles formed before and after encapsulation of GW3965 were measured by DLS.

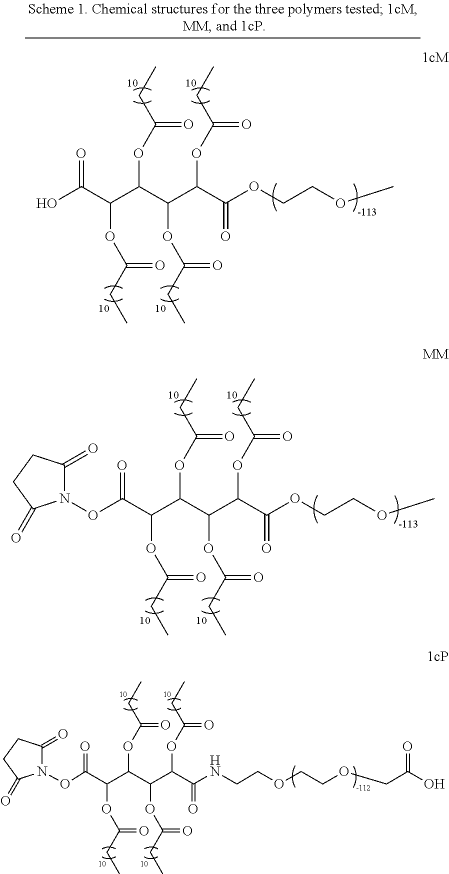

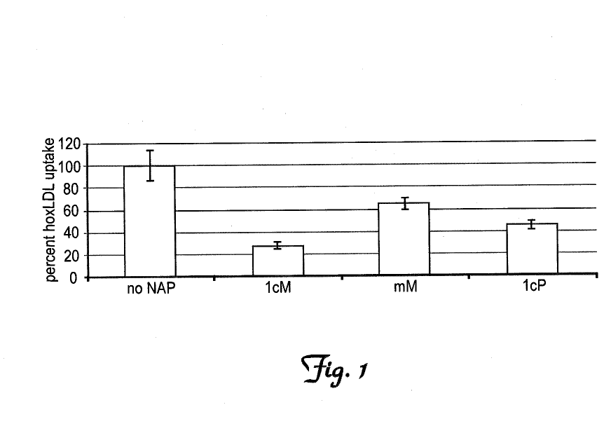

All three of the NAPS tested were able to significantly inhibit hoxLDL uptake by human macrophage cells, but the 1 cM NAP was able to inhibit uptake significantly more the other NAPs, leading to only 27% hoxLDL uptake in comparison to the condition with no NAPs present. This is in comparison to a 64% and 45% inhibition by the mM and 1 cP NAPs respectively (FIG. 1).

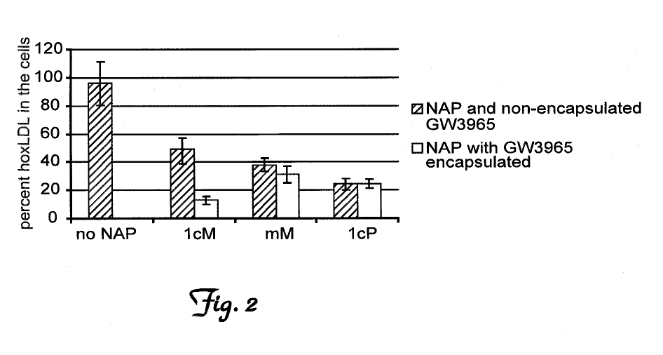

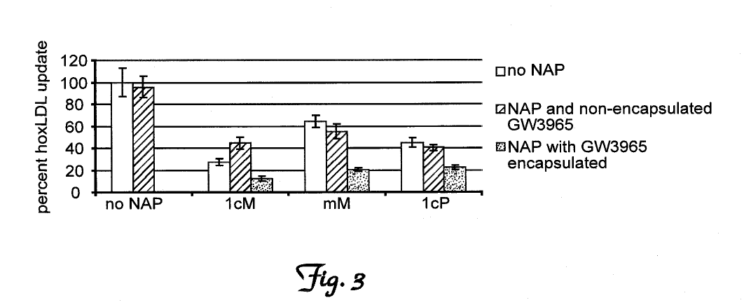

The effect on efflux of hoxLDL from macrophage cells when GW3965 was administered was then investigated. The delivery of a small concentration of LXR ligand has no significant effect of hoxLDL concentration within the cells, leading to only 4% less hoxLDL within the cells then a condition in which nothing was administered to the cells. But when the ligand is delivered with the NAPs there is an increase in cholesterol efflux. It was further demonstrated that 86% of cholesterol will be released from the cells when the ligand is delivered encapsulated within the 1 cM anionic nanoparticle, significantly more then when GW3965 is encapsulated within mM and 1 cP which leads to 69% and 76% less hoxLDL respectively (FIG. 2).

The ability of a composition of the invention to inhibit hoxLDL uptake as well as cause hoxLDL efflux was then investigated. This two pronged approach leads to significant inhibition of hoxLDL within cells, down to 12% of that of cells without the NAPs or the LXR ligand. This is significantly less hoxLDL then any other condition, but the mM and 1 cP NAPs with ligand encapsulation were also able to significantly down-regulate the hoxLDL content of cells, down to 21% and 23% of cells without NAPs or ligand (FIG. 3).

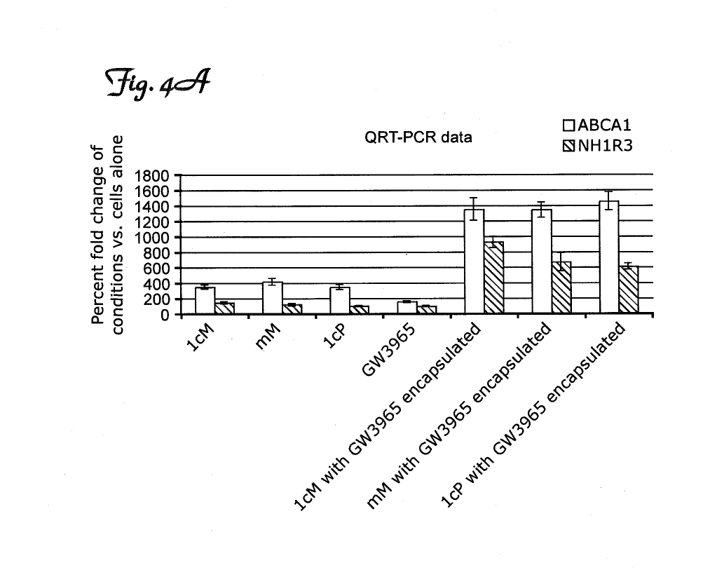

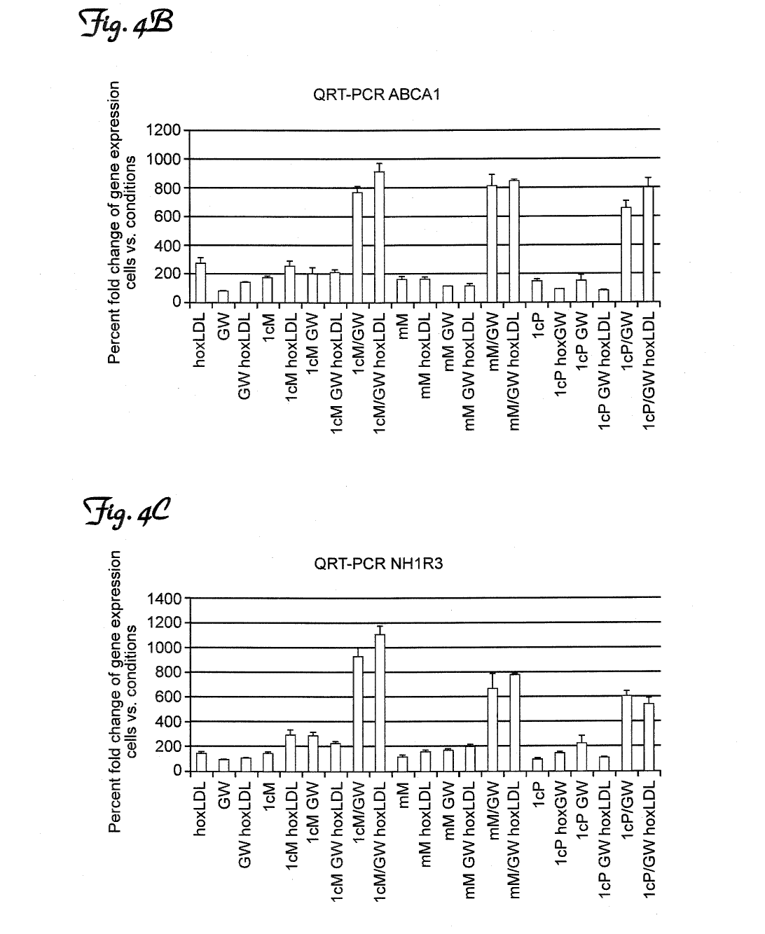

The effects of the delivery of GW3965 to the cells on gene expression was explored to determine the extent of change in the cells. It was discovered that when the GW3965 was encapsulated within NAPs and delivered to the cells there was an increase in gene expression that was not seen when GW3965 was not encapsulated. Two of the genes that we choose to investigate are ABCA1, a cell associated protein that leads to cholesterol efflux, and NH1R3, associated with LXRalpha. It was shown that ABCA1 was upregulated to similar extent independent of the nature of NAP that was utilized to deliver the ligand, while NH1R3 was upregulated more by 1 cM then by the other two nanoparticles. To ensure that the interactions of hoxLDL did not alter the trends that were observed, gene expression was also examined for all conditions that were examined for the hoxLDL internalization studies. It was shown that the presence of hoxLDL caused no significant alteration in gene expression upregulation by the NAPs and ligand (FIGS. 4A, B, C).

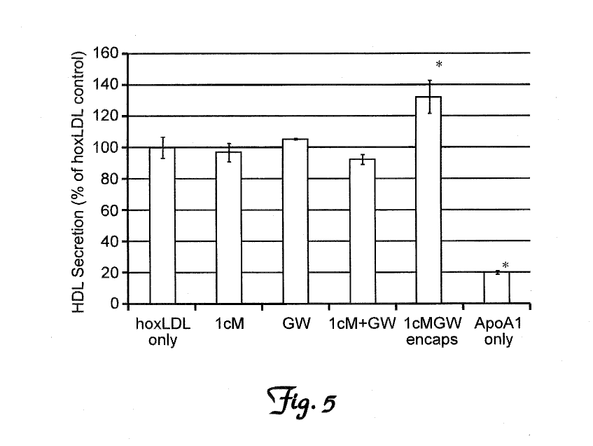

Because GW3965 encapsulated within 1 cM significantly up-regulated LXR related genes, studies were carried out to determine whether the up-regulation results in an increased ability to repackage cellular cholesterol as HDL in THP-1 macrophages. The ability of encapsulated GW3965 to enhance cholesterol efflux was examined in human THP-1 macrophages pre-incubated with hoxLDL (30 μg/ml). The addition of ApoAI protein to the cells served to promote cholesterol efflux, and in the absence of hoxLDL minimally increased cholesterol efflux, as shown in FIG. 5. The level of HDL secreted in THP-1 macrophages exposed to hoxLDL alone was normalized to 100 percent and the amount of HDL secreted in the conditions containing 1 cM, GW3965, or 1 cM with GW3965 were not significantly different from the 100 percent baseline. However, the cells incubated with hoxLDL and GW3965 encapsulated within 1 cM exhibited an enhanced cholesterol efflux. By presenting the GW3965 to the cells through 1 cM NAP encapsulation the total HDL secretion was increased by 35%.

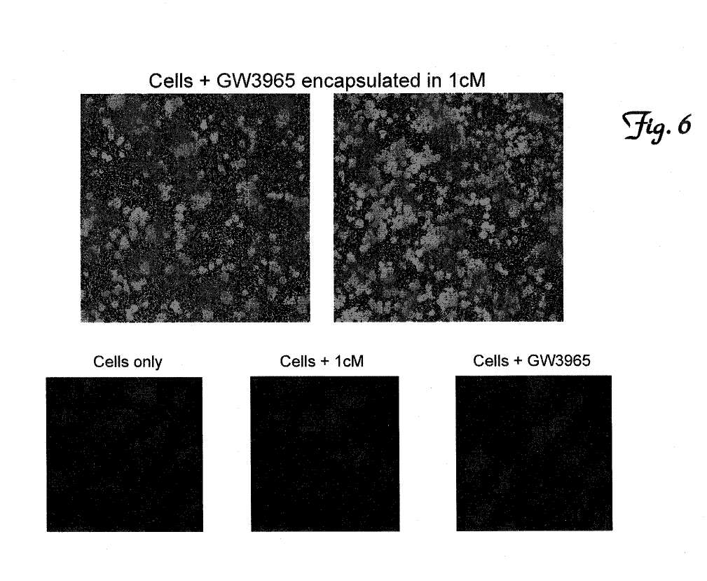

In light of the results indicating the enhanced ability of encapsulated GW3965 to reduce total hoxLDL accumulation, multiphoton images were taken using a Leica TCS SP2 system (Leica Microsystems, Inc., Exton, Pa.) in order to visually confirm the delivery and internalization of GW3965 to THP-1 macrophage cells incubated for 5 hr with 10⁻⁶ M of each polymer. GW3965, which emits around 235 nm, was seen most strongly in the conditions containing GW3965 encapsulated within the 1 cM NAP (FIG. 6). The condition containing GW3965 without 1 cM delivery shows minimal uptake by the macrophages compared to the control conditions of macrophages alone and macrophages with 1 cM.

### Discussion

The above results demonstrate the beneficial role of polymer-based targeted drug delivery for controlled cholesterol accumulation within immune blood cells that are critical for foam cell formation and atherosclerosis. This was accomplished through the dual functionality of the polymers: First, through scavenger receptor targeting, which allows for blocking of exogenous oxidized LDL uptake, and second by intracellular delivery of the drug cargo, which, in this instance, was demonstrated for a nuclear targeted drug for reversal of cholesterol accumulation. The combination of these two effects resulted in maximal reduction of cholesterol accumulation, which has important implications for the use of such polymers for targeting of high risk sites for atherosclerotic lesions and plaques.

Previous studies that have examined cardiovascular diseases' association with LXR have focused on either cells or mice that lack LXR-alpha and/or LXR-beta. The use of the THP-1 human monocyte derived macrophage cells showed that the encapsulation of the LXR-ligand, GW3965, by the nanoparticle led to a large increase in efficacy of the drug. At the small concentration of 1*10⁻⁸M, GW3965 was not effective in efflux or inhibition of influx of hoxLDL when delivered directly (without the use of polymer carriers) to the cells. In contrast there was a large decrease in the highly oxidized LDL (hoxLDL0 within cells when similar low concentrations of drug were delivered through encapsulation by nanoparticles. The implication of this finding is that, in practice, the compositions of the invention may allow very low quantities of the drug to be administered to elicit the requisite therapeutic benefits. There are no other studies that demonstrate heightened potency with these cardiovascular drugs at lower concentrations through the use of carrier-assisted delivery.

A study by Albers et al. showed the upregulation of LXRalpha and ABCA1 in HepG2 cells, but a delivery of GW3965 at concentrations as high as 1*10⁻⁶M was only able to cause up to a 3-fold (LXRalpha) and 6-fold (ABCA1) increase (Albers M., et al., Journal of Biological Chemistry 281(8): 4920-4930), as opposed to the 9-fold and 15-fold increase that the ligand initiated when it was delivered within the polymeric nanoparticles in a composition of the invention. It is hypothesized that the internalization of the nanoparticles, hence ligand, by macrophage scavenger receptors leads to an increased delivery of GW3965 to the nuclear membrane.

By presenting GW3965 to the cells through 1 cM nanoparticle encapsulation, enhanced cholesterol efflux was observed and the total high density lipoprotein (HDL) secretion was increased by 35% compared to the no intervention control. This is a considerable finding as HDL plays a key role in the evolution of atherosclerosis. While 1 cM alone can significantly reduce the accumulation of hoxLDL and foam cell formation within macrophages, the ability to both reduce accumulation and enhance the efflux of cellular cholesterol results in a comprehensive treatment option. By utilizing the 1 cM as a delivery vehicle for GW3965 the ability to amplify the production of HDL and provide a complementary approach to manage the progression of atherosclerosis was demonstrated.

To visually confirm the delivery and internalization of GW3965 to THP-1 macrophage cells, multiphoton images were taken which illustrated the ability of the 1 cM as a delivery vehicle. GW3965 was seen most strongly in the conditions containing GW3965 encapsulated within 1 cM while the condition containing GW3965 without 1 cM delivery shows minimal uptake by the macrophages compared to the control conditions. The 1 cM has been shown previously to bind and be internalized via the macrophage scavenger receptors SR-A and CD36. It is possible that the enhanced internalization is due to the binding between the anionic carboxylate groups of the 1 cM and the positive pocket of residues on the SR-A scavenger receptor. This enhanced delivery allows for use of lower doses of GW3965. Further, this may allow for targeted delivery to cells expressing high levels of SR-A and the related receptors, which are upregulated at the sites of atherosclerotic lesions and vulnerable atherosclerotic plaques.

All publications, patents, and patent documents, particularly all relevant sections of the documents mentioned in this patent are incorporated by reference herein, as though individually incorporated by reference. The invention has been described with reference to various specific and preferred embodiments and techniques. However, it should be understood that many variations and modifications may be made while remaining within the spirit and scope of the invention.

1. A composition comprising a cardiovascular agent and one or more compounds of formula (I):
A-X—Y—Z—R₁ (I)
wherein:
A is H, HOSO₂—O—, —C(═O)N(H)—Rₐ, or —C(═O)O—Rₐ
Rₐ is H, R_{b}, or a (C₁-C₆)alkyl chain, wherein one or more carbon atoms in the alkyl chain is optionally replaced with NH, which chain is optionally substituted with one or more carboxy, sulfo, HOSO₂—O—, amino, or R_{b;}
R_{b} is 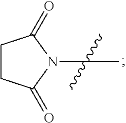
X is a polyol, wherein one or more polyol hydroxyls are substituted by acyl;
Y is —C(═O)—, —C(═S)—, or is absent;
Z is O, S or NH; and
R₁ is a polyether.

2. The composition of claim 1 wherein the cardiovascular agent is an agent that modulates cholesterol metabolism.

3. The composition of claim 1 wherein the cardiovascular agent is a liver x receptor agonist.

4. The composition of claim 1 wherein the cardiovascular agent is Diepoxycholesterol, T0901317, GW3965, or 24(S),25-Epoxycholesterol.

5. The composition of claim 1 wherein the cardiovascular agent is 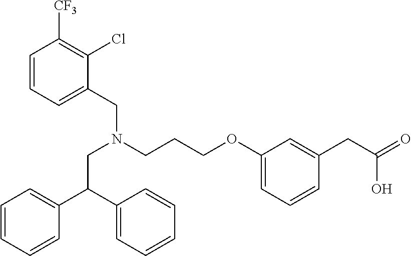
or a pharmaceutically acceptable salt thereof.

6. The composition of claim 1 wherein A is HOSO₂—O—.

7. The composition of claim 1 wherein A is —C(═O)N(H)—Rₐ.

8. The composition of claim 1 wherein A is —C(═O)O—Rₐ.

9. The composition of claim 1 wherein A is C(═O)OH.

10. The composition of claim 1 wherein A is

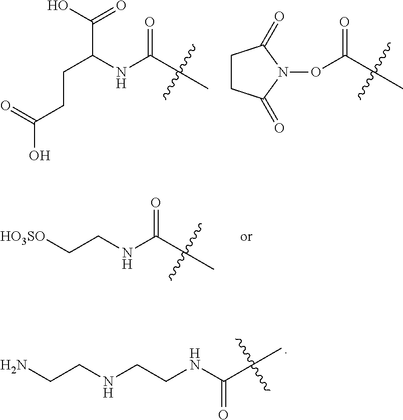

11. The composition of claim 1, wherein the polyol is a (C₂-C₂₀) alkyl polyol.

12. The composition of claim 1, wherein the polyol comprises about 2 to about 20 hydroxyl groups.

13. The composition of claim 1, wherein the polyol is substituted with one or more acyl.

14. The composition of claim 1, wherein the polyol comprises a mono- or dicarboxylic (C₂-C₂₀) alkyl polyol substituted with about 1 to about 10 hydroxyl(s).

15. The composition of claim 1, wherein the polyol comprises one or more of mucic acid, malic acid, citromalic acid, alkylmalic acid, hydroxy glutaric acid derivatives, alkyl glutaric acids, tartaric acid, or citric acid.

16. The composition of claim 1, wherein the polyol comprises one or more of 2,2-(bis(hydroxymethyl)propionic acid, tricine, or a saccharide.

17. The composition of claim 1, wherein the polyether comprises about 2 to about 150 alkylene oxide units.

18. The composition of claim 1, wherein each alkylene oxide unit comprises straight or branched (C₂-C₄) alkylene oxide.

19. The composition of claim 1, wherein the polyether comprises an alkoxy-terminal group or a carboxy terminal group.

20. The composition of claim 1, wherein the polyether is linked to the polyol through a linker comprising an ester, thioester, or amide linkage.

21. The composition of claim 1, wherein the polyether comprises the chemical formula
R₅—(R₆—O—)ₐ—R₆-Q- (II),
wherein:
R₅ comprises straight or branched (C₁-C₂₀) alkyl, —OH, —OR₇, —NH₂, —NHR₇, —NHR₇R₈, —CO₂H, —SO₃H, —OSO₃H, —CH₂—OH, —CH₂—OR₇, —CH₂—O—CH₂—R₇, —CH₂—NH₂, —CH₂—NHR₇, —CH₂—NR₇R₈, —CH₂CO₂H, —CH₂SO₃H, or —O—C(═O)—CH₂—CH₂—C(═O)—O—;
R₆ comprises straight or branched divalent (C₂-C₁₀) alkylene;
each R₇ and R₈ comprises, independently, straight or branched (C₁-C₆) alkylene;
Q comprises —O—, —S—, or —NR₇; and
a comprises an integer of about 2 to about 110, inclusive.

22. The composition of claim 1, wherein the polyether comprises a polyethylene glycol comprising a methoxy terminal group or a carboxy terminal group.

23. The composition of claim 1, wherein the fatty acid(s) comprise(s) (C₂-C₂₄) fatty acid(s).

24. The composition of claim 1, wherein the fatty acid(s) comprise(s) one or more of caprylic, capric, lauric, myristic, myristoleic, palmitic, palmitoleic, stearic, oleic, linoleic, arachidic, behenic, or erucic acid.

25. The composition of claim 1 wherein the compound of formula (I) is 1 cP, 1 cM, or MM:

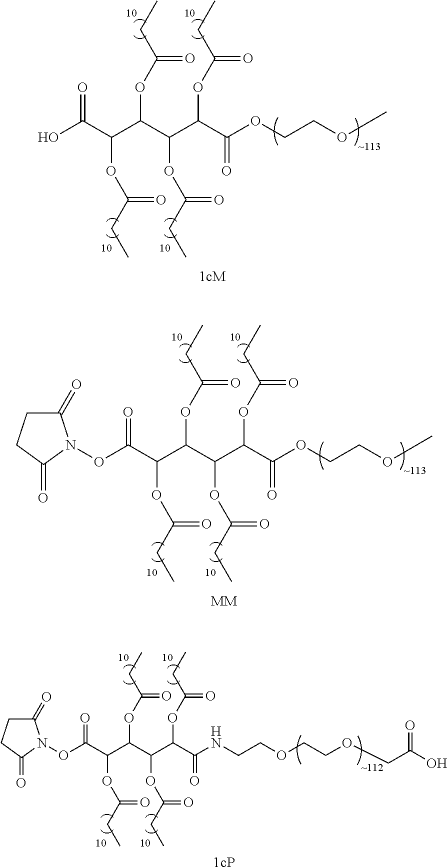

26. The composition of claim 1, which comprises a plurality of compounds of formula (I) that form a nanoscale assembly or particulate formulation.

27. The composition of claim 1 wherein the cardiovascular agent is surrounded or partially surrounded by at least one compound of formula (I).

28. The composition of claim 1 that further comprises a pharmaceutically acceptable diluent or carrier.

29. A method for inhibiting atherosclerosis or atherosclerotic development, in an animal comprising administering a composition as described in claim 1 to the animal.

30-31. (canceled)

32. A method for treating a cardiovascular disease in an animal comprising administering a composition as described in claim 1 to the animal.

33-40. (canceled)
